(19) United States
(12) Patent Application Publication
(10) Pub. No.: US 2011/0301204 A1
(43) Pub. Date: Dec. 8, 2011

(54) ADMINISTRATION REGIME FOR NITROCATECHOLS

(75) Inventors: José Luis de Almeida, Chave - Arouca (PT); David Alexander Learmonth, Alfena (PT); Patricio Manuel Vieira Araújo Soares da Silva, Porto (PT)

(73) Assignee: BIAL - PORTELA & CA., S.A., S. Mamede do Coronado (PT)

(21) Appl. No.: 13/002,287

(22) PCT Filed: Jul. 29, 2009
(86) PCT No.: PCT/PT2009/000044

§ 371 Date: Jul. 7, 2011

Related U.S. Application Data
(60) Provisional application No. 61/137,248, filed on Jul. 29, 2008.

(51) Int. Cl. A61K 31/4439; A61P 25/16; A61P 25/14; C07D 413/04; A61P 25/02

(52) U.S. Cl. 514/340; 546/269.4

(57) ABSTRACT

The present invention relates to novel dosage regimens for compounds of formula I: where the substituents are as defined in the specification.

This invention relates to novel substituted nitrocatechols and to their use in the treatment of central and peripheral nervous system disorders according to a specified dosing regimen.

The rationale for the use of COMT inhibitors as adjuncts to L-DOPA/aromatic L-amino acid decarboxylase inhibitor (AADCi) therapy is based on their ability to reduce metabolic O-methylation of L-DOPA to 3-O-methyl-L-DOPA (3-OMD). The duration of L-DOPA-induced clinical improvement is brief as a result of the short in vivo half-life of L-DOPA which contrasts with the long half-life of 3-OMD. Additionally, 3-OMD competes with L-DOPA for transport across the blood-brain barrier (BBB), which means that only a very limited amount of an orally administered dose of L-DOPA actually reaches the site of action, i.e. the brain. Commonly, within only a few years of starting L-DOPA therapy with the usual dosage regime, L-DOPA-induced clinical improvement declines at the end of each dose cycle, giving rise to the so-called ‘wearing-off’ pattern of motor fluctuations. A close relationship between the ‘wearing-off’ phenomenon and accumulation of 3-OMD has been described (Tohgi, H., et al., Neurosci. Letters, 132:19-22, 1992). It has been speculated that this may result from impaired brain penetration of L-DOPA due to competition for the transport system across the BBB with 3-OMD (Reches, A. et al., Neurology, 32:887-888, 1982) or more simply that there is less L-DOPA available to reach the brain (Nutt, J. G., Fellman, J. H., Clin. Neuropharmacol., 7:35-49, 1984). In effect, COMT inhibition protects L-DOPA from O-methylation metabolic breakdown in the periphery, such that with repeated doses of L-DOPA, the mean plasma L-DOPA concentration is raised. In addition to reduced competition for transport into the brain, a significantly greater percentage of the orally administered dose of L-DOPA is able to reach the site of action. Thus COMT inhibition serves to increase the bioavailability of L-DOPA and the duration of antiparkinsonian action is prolonged with single doses of L-DOPA (Nutt, J. G., Lancet, 351:1221-1222, 1998).

The most potent COMT inhibitors reported thusfar are 3,4-dihydroxy-4′-methyl-5-nitrobenzophenone (Tolcapone, Australian pat. AU-B-69764/87) and (E)-2-cyano-N,N-diethyl-3-(3,4-dihydroxy-5-nitrophenyl)acrylamide (Entacapone, German pat. DE 3740383 A1).

Although sharing essentially the same pharmacophore, tolcapone differs from entacapone in that it easily enters the central nervous systems (CNS) and is able to inhibit cerebral COMT as well as peripheral COMT. Shortly after its launch, tolcapone was withdrawn from the market after several cases of hepatotoxicity were reported including three unfortunate deaths from fatal fulminant hepatitis. Today tolcapone can only be used in Parkinsonian patients who are unresponsive to other treatments and only with regular monitoring of liver function, which is expensive and inconvenient for the patient. Although the actual mechanistic causes of the liver toxicity associated with tolcapone are not fully understood, in vitro studies have shown that tolcapone may be reduced metabolically to reactive intermediates and it has been speculated that these may form covalent adducts with hepatic proteins resulting in hepatocellular injury (Smith, K. S. et al, Chem. Res. Toxicol., 16:123-128, 2003).

Entacapone on the other hand, although sharing the same nitrocatechol pharmacophore with tolcapone, is not associated with liver toxicity and is generally regarded as a safe drug. Unfortunately however, entacapone is a significantly less potent COMT inhibitor than tolcapone and has a much shorter in-vivo half-life. This means that entacapone has a very limited duration of effect and as a consequence, the drug must be administered in very high doses with every dose of L-DOPA taken by the patient. As such, the clinical efficacy of entacapone has been questioned—indeed a recent study (Parashos, S. A. et al., Clin. Neuropharmacol., 27 (3): 119-123, 2004) revealed that the principal reason for discontinuation of entacapone treatment in Parkinson's disease patients was a perceived lack of efficacy.

Furthermore, the relatively short in-vivo half-life of known COMT inhibitors requires continuous treatment regimens normally involving the administration of several doses a day which many patients find to be burdensome. For example, tolcapone has to be administered three times a day. This factor can therefore interfere with patient compliance and quality of life.

Accordingly, there is still a need for COMT inhibitors exhibiting balanced properties of bioactivity, bioavailability and safety. In particular, there is a need for COMT inhibitors having a long in-vivo half-life and, thus, a prolonged action on COMT enabling fewer dosages to obtain the desired therapeutic effect.

We have now surprisingly found that, despite having a relatively short half life, compounds of general formula I are very potent COMT inhibitors endowed with exceptionally long duration of action compared to COMT inhibitors in the prior art.

Compounds of general formula I also markedly enhance the bioavailability of L-DOPA and increase the delivery of L-DOPA to the brain. The compounds significantly augment the levels of dopamine in the brain over a long period of time.

Even more surprisingly, the increased levels of L-DOPA are maintained steady over extended periods of time. These sustained effects upon both COMT activity and L-DOPA bioavailability after the administration of compounds of general formula I are markedly greater than those observed with tolcapone, the only COMT inhibitor thusfar known to be endowed with a reasonably long duration of action. (Tolcapone has a terminal half life of around 2 hours and must be administered around 3 times per day.) Furthermore, compounds of general formula I produce a steady increase in L-DOPA delivery to the brain over extended periods of time, which contrasts with that observed with tolcapone, which is prone to induce marked oscillations in the brain delivery of L-DOPA. Thus compounds of general formula I are more likely to be endowed with therapeutic advantages due to sustained constant elevation of L-DOPA levels whilst the use of tolcapone is likely to induce undesirable side-effects such as dyskinesia due to abrupt increases and decreases in L-DOPA levels.

Compounds of general formula I are compounds having the following formula

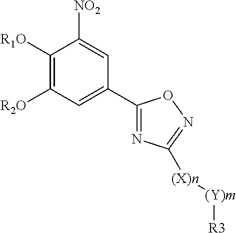

where R₁ and R₂ are the same or different and signify hydrogens, groups hydrolysable under physiological conditions, or optionally substituted alkanoyls or aroyls; X signifies a methylene group; Y represents O, S or NH; n represents 0, 1, 2 or 3; m represents 0 or 1; R₃ signifies a pyridine N-oxide group according to the formula A, B, or C, which is connected as indicated by the unmarked bond:

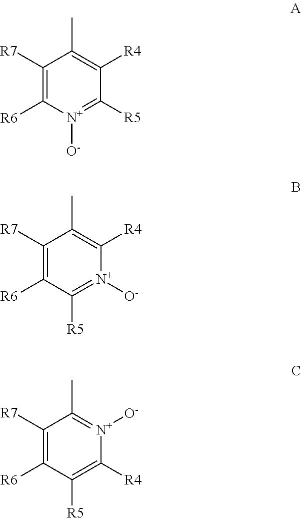

where R₄, R₅, R₆ and R₇ are the same or different, and signify hydrogen, alkyl, thioalkyl, alkoxy, aryloxy, thioaryl, alkanoyl, aroyl, aryl, amino, alkylamino, dialkylamino, cycloalkylamino, heterocycloalkylamino, alkylsulphonyl, arylsulphonyl, halogen, haloalkyl, trifluoromethyl, cyano, nitro or heteroaryl; or two or more of R₄, R₅, R₆ and R₇ taken together signify aliphatic or heteroaliphatic rings or aromatic or heteroaromatic rings; the term ‘alkyl’, including its variant ‘alk-’ in terms such as ‘alkoxy’, ‘alkanoyl’ mean carbon residues, straight or branched, containing from one to six carbon atoms; the term ‘aryl’ means a phenyl or naphthyl group; the term ‘heterocycloalkyl’ represents a four to eight-membered cyclic ring optionally incorporating at least one atom of oxygen, sulphur or nitrogen; the term ‘heteroaryl’ represents a five or six-membered ring incorporating at least one atom of sulphur, oxygen or nitrogen; the term ‘halogen’ represents fluorine, chlorine, bromine or iodine; and if R₄, R₅, R₆ and R₇ represent alkyl or aryl they are optionally substituted by one or more hydroxy, alkoxy or halogen groups; or a pharmaceutically acceptable salt or ester thereof.

Preferably, R₄, R₅, R₆ and R₇ independently from each other represent hydrogen, C₁-C₆-alkyl, C₁-C₆-thioalkyl, C₁-C₆-alkoxy, C₆-C₁₀-aryloxy, C₆-C₁₀-thioaryl, C₁-C₆-alkanoyl, C₁-C₁₁-aroyl, amino, C₁-C₆-alkylamino, di-C₁-C₆-alkylamino, C₃-C₁₂-cycloalkylamino, C₄-C₈-heterocycloalkylamino, C₁-C₆-alkylsulphonyl, C₆-C₁₀-arylsulphonyl, halogen, C₁-C₆-haloalkyl, trifluoromethyl, cyano, nitro or heteroaryl.

When R₄, R₅, R₆ and/or R₇ represent C₁-C₆-alkyl residues, preferably R₄, R₅, R₆ and/or R₇ represent methyl, ethyl, n-propyl, isopropyl, n-butyl, sec-butyl, tert-butyl, pentyl, or hexyl.

When R₄, R₅, R₆ and/or R₇ represent C₁-C₆-thioalkyl residues, preferably R₄, R₅, R₆ and/or R₇ represent thiomethyl, thioethyl, thio-n-propyl, thio-isopropyl, thio-n-butyl, thio-n-pentyl, or thio-n-hexyl.

When R₄, R₅, R₆ and/or R₇ represent C₁-C₆-alkoxy residues, preferably R₄, R₅, R₆ and/or R₇ represent methoxy, ethoxy, n-propoxy, isopropoxy, n-butoxy, sec-butoxy or tert-butoxy.

When R₄, R₅, R₆ and/or R₇ represent C₆-C₁₀-aryloxy residues, preferably R₄, R₅, R₆ and/or R₇ represent phenoxy or naphthoxy.

When R₄, R₅, R₆ and/or R₇ represent C₆-C₁₀-thioaryl residues, preferably R₄, R₅, R₆ and/or R₇ represent thiophenyl or thionaphthyl.

When R₄, R₅, R₆ and/or R₇ represent C₁-C₆-alkanoyl residues, preferably R₄, R₅, R₆ and/or R₇ represent methanoyl, ethanoyl, propanoyl or butanoyl.

When R₄, R₅, R₆ and/or R₇ represent C₇-C₁₁-aroyl residues, preferably R₄, R₅, R₆ and/or R₇ represent benzoyl or naphthoyl.

When R₄, R₅, R₆ and/or R₇ represent C₁-C₆-alkylamino residues, preferably R₄, R₅, R₆ and/or R₇ represent methylamino, ethylamino, n-propylamino, isopropylamino or n-butylamino.

When R₄, R₅, R₆ and/or R₇ represent di-C₁-C₆-alkylamino residues, preferably R₄, R₅, R₆ and/or R₇ represent dimethylamino, diethylamino, di-n-propylamino, di-n-butylamino, di-isopropylamino, methylethylamino, methylpropylamino or ethylpropylamino.

When R₄, R₅, R₆ and/or R₇ represent C₃-C₁₂-cycloalkylamino residues, preferably R₄, R₅, R₆ and/or R₇ represent pyrrolidino, piperidino, cyclohexylamino or dicyclohexylamino.

When R₄, R₅, R₆ and/or R₇ represent C₄-C₈-heterocycloalkylamino residues, preferably R₄, R₅, R₆ and/or R₇ represent morpholino, 2,6-dimethylmorpholino, 3,5-dimethylmorpholino, piperazino, N-methylpiperazino or N-ethylpiperazino.

When R₄, R₅, R₆ and/or R₇ represent C₁-C₆-alkylsulphonyl or C₆-C₁₀-arylsulphonyl residues, preferably R₄, R₅, R₆ and/or R₇ represent methylsulfonyl, ethylsulfonyl, phenylsulfonyl, or tolylsulfonyl.

When R₄, R₅, R₆ and/or R₇ represent halogen residues, preferably R₄, R₅, R₆ and/or R₇ represent chloro, bromo, iodo or fluoro.

When R₄, R₅, R₆ and/or R₇ represent C₁-C₆-haloalkyl residues, preferably R₄, R₅, R₆ and/or R₇ represent chloromethyl, fluoromethyl, dichloromethyl, difluoromethyl, trichloromethyl or trifluoromethyl.

When R₄, R₅, R₆ and/or R₇ represent heteroaryl residues, preferably R₄, R₅, R₆ and/or R₇ represent pyridyl, pyrimidyl, isoxazolyl, oxazolyl, isoxadiazolyl, oxadiazolyl, triazolyl or tetrazolyl.

When two or more of residues R₄, R₅, R₆ and R₇ taken together represent aliphatic or heteroaliphatic rings or aromatic or heteroaromatic rings, the two or more residues preferably represent aliphatic or heteroaliphatic rings or aromatic or heteroaromatic rings. Preferred combined residues are indolizinyl, isoindolyl, indolyl, indazolyl, purinyl, quinolizinyl, naphthyridinyl, isoquinolyl and quinolyl.

Where they represent aryl or alkyl, the above substituents R₄, R₅, R₆ and R₇ may optionally be substituted one or more times by hydroxy, alkoxy or halogen groups.

Details of the preparation of compounds of general formula I can be found in WO2007/013830A1.

The bioavailability, bioactivity, safety profile and other related properties known in the art (e.g. blood-brain-barrier permeability) can be routinely optimized by the skilled person on basis of the teaching of the present application by varying substituents R₁-R₇ of the above general formula I in order to obtain a desirable balanced mix of properties.

The compounds of general formula I may also be present in the form of pharmacologically acceptable salts or esters thereof. Suitable pharmaceutically acceptable counter ions are known to the art.

It is also possible to use prodrugs of compounds of the general formula I in order to alter the therapeutic profile of the active compound.

In the following description of medical indications, treatments and dosing regimens for pharmaceutical compositions containing compounds according to general formula I of the invention, the most preferred example of a compound according to the general formula I is 5-[3-(2,5-dichloro-4,6-dimethyl-1-oxy-pyridin-3-yl)-[1,2,4]oxadiazol-5-yl]-3-nitrobenzene-1,2-diol, henceforth designated as compound A, and its pharmacologically acceptable salts and esters. The half life of compound A is relatively short given its long duration of action.

Other preferred compounds of the above general formula (I) for use in the subsequent medical indications, treatments and dosing regimens include 3-(3-(3,4-dihydroxy-5-nitrophenyl)-1,2,4-oxadiazol-5-yl)-4-(trifluoromethyl)pyridine-1-oxide, 2-chloro-3-(3-(3,4-dihydroxy-5-nitrophenyl)-1,2,4-oxadiazol-5-yl)-4,6-dimethylpyridine-1-oxide, 3-(3-(3,4-dihydroxy-5-nitrophenyl)-1,2,4-oxadiazol-5-yl)-2-methyl-6-(trifluoromethyl)pyridine-1-oxide, 5-(3-(3,4-dihydroxy-5-nitrophenyl)-1,2,4-oxadiazol-5-yl)-2-(trifluoromethyl)pyridine-1-oxide, 5-(3-(3,4-dihydroxy-5-nitrophenyl)-1,2,4-oxadiazol-5-yl)-2-methyl-4-(trifluoromethyl)pyridine-1-oxide, 3-(3-(3,4-dihydroxy-5-nitrophenyl)-1,2,4-oxadiazol-5-yl)-2,6-dimethyl-4-(trifluoromethyl)pyridine-1-oxide, 3,5-dichloro-4-(3-(3,4-dihydroxy-5-nitrophenyl)-1,2,4-oxadiazol-5-yl)pyridine-1-oxide, 3-(3-(3,4-dihydroxy-5-nitrophenyl)-1,2,4-oxadiazol-5-yl)-6-methyl-2-phenyl-4-(trifluoromethyl)pyridine-1-oxide, 2-bromo-3-(3-(3,4-dihydroxy-5-nitrophenyl)-1,2,4-oxadiazol-5-yl)-4,5,6-trimethylpyridine-1-oxide, 2-chloro-3-(3-(3,4-dihydroxy-5-nitrophenyl)-1,2,4-oxadiazol-5-yl)-4,5,6-trimethylpyridine-1-oxide, 3-(3-(3,4-dihydroxy-5-nitrophenyl)-1,2,4-oxadiazol-5-yl)-2-(trifluoromethyl)pyridine-1-oxide, 2,5-dichloro-3-(3-(3,4-dihydroxy-5-nitrophenyl)-1,2,4-oxadiazol-5-yl)-4,6-dimethylpyridine-1-oxide, 3-(3-(3,4-dihydroxy-5-nitrophenyl)-1,2,4-oxadiazol-5-yl)-5-(trifluoromethyl)pyridine-1-oxide, 3-(3-(3,4-dihydroxy-5-nitrophenyl)-1,2,4-oxadiazol-5-yl)-2-fluoropyridine-1-oxide, 4-(3-(3,4-dihydroxy-5-nitrophenyl)-1,2,4-oxadiazol-5-yl)-2-fluoropyridine-1-oxide, 2-(3-(3,4-dihydroxy-5-nitrophenyl)-1,2,4-oxadiazol-5-yl)-6-fluoropyridine-1-oxide, 2-chloro-3-(3-(3,4-dihydroxy-5-nitrophenyl)-1,2,4-oxadiazol-5-yl)-6-methylpyridine 1-oxide, 2-bromo-3-(3-(3,4-dihydroxy-5-nitrophenyl)-1,2,4-oxadiazol-5-yl)-6-methylpyridine-1-oxide, and 2-bromo-5-chloro-3-(3-(3,4-dihydroxy-5-nitrophenyl)-1,2,4-oxadiazol-5-yl)-4,6-dimethylpyridine-1-oxide and their pharmacologically acceptable salts or esters.

The present invention relates to the use of the compounds of general formula I, their pharmaceutically acceptable salts or esters for prevention or treatment of certain pathological states, especially in humans, (e.g. central and peripheral nervous system disorders) and to preparation of pharmaceutical compositions containing them.

For the preparation of pharmaceutical compositions of compounds of general formula I, inert pharmaceutically acceptable carriers are admixed with the active compounds. The pharmaceutically acceptable carriers may be solid or liquid. Solid form preparations include powders, tablets, dispersible granules and capsules. A solid carrier can be one or more substances which may also act as diluent, flavouring agent, solubiliser, lubricant, suspending agent, binder, glidant, or disintegrant; it may also be an encapsulating material.

Preferably the pharmaceutical composition is in unit dosage form, e.g. a packaged preparation, the package containing discrete quantities of the preparation, for example packaged tablets, capsules and powders in vials or ampoules.

Preferably, the treated pathological states are central and peripheral nervous system-associated disorders of humans, and in particular those which benefit from administration of a COMT inhibitor. Preferably, the disorders are movement disorders including disorders involving parkinsonism, Parkinson's Disease, and restless leg syndrome. The most preferred central and peripheral nervous system associated disorder is Parkinson's Disease.

As used herein, the term treatment and variations such as ‘treat’ or ‘treating’ refer to any regime that can benefit a human or non-human animal. The treatment may be in respect of an existing condition or may be prophylactic (preventative treatment). Treatment may include curative, alleviation or prophylactic effects, such effects relating to one or more of the symptoms associated with the central and peripheral nervous system-associated disorders.

The compounds of the general formula I are preferably used for the preparation of a medicament for the prevention or treatment of central and peripheral nervous system associated disorders according to a specified dosing regimen.

Suitable dosing regimens comprise regimens having a dosing periodicity ranging from about twice a day to about once every seven days.

Preferably, the dosing periodicity is selected from once every third day to once weekly, i.e. administration about once every 3^{rd}, 4^{th}, 5^{th}, 6^{th} or 7^{th} day.

Suitable non-limiting starting points for dosing intervals comprise the morning, mid-day, noon, afternoon, evening, and midnight.

As used herein, the term ‘effective daily dose’ is the effective daily amount of compound administered when administered according to the dosing periodicity.

In the present invention, effective daily doses of compounds of general formula I are in the range of about 1 to about 900 mg/day, more preferably about 5 to about 400 mg/day, even more preferably about 25 to about 300 mg/day, even more preferably about 70 to about 200 mg/day and most preferably about 120 to about 150 mg/day.

As used herein, the term “dosage unit” refers to the amount of compound administered in each dosing periodicity.

It is preferred that individual dosage units of compounds of general formula I are in the range of about 1 to about 2400 mg, more preferably about 10 to about 1200 mg, even more preferably about 25 to about 800 mg, even more preferably about 50 to about 400 mg, and most preferably about 100 to about 200 mg.

Preferably the subject being treated with the compound of general formula I is also receiving therapy with a dopamine (DOPA) precursor and/or an AADCi.

Typical DOPA precursors include L-DOPA.

Suitable aromatic L-amino acid decarboxylase inhibitors include benserazide and carbidopa.

The compounds of general formula I, DOPA precursor and AADCi may be administered separately or in any combination. They may be administered concomitantly (for example, simultaneously) or sequentially, and with the same or differing dosing periodicity. For example, the compounds of the general formula I can be concomitantly or sequentially administered with DOPA precursor. In case of concomitant administration it is also possible to combine both or all active ingredients in one unit dosage form.

According to another aspect of the present invention there is provided a method of treating at least one pathological state in a patient in need thereof comprising administering about once every third day to about once weekly a pharmacologically effective dose of a compound of general formula I as defined above to the patient.

According to another aspect of the invention there is provided a method for reducing COMT inhibition in a subject over 3 to 7 days, comprising administering, about once every third day to about once weekly, an effective dose of a compound of general formula I as defined above to the subject.

According to another aspect of the invention there is provided a method for increasing levels of L-DOPA in the brain of a subject over 3 to 7 days, comprising administering, about once every third day to about once weekly, an effective dose of a compound of general formula I as defined above to the subject.

According to another aspect of the invention there is provided a method for increasing levels of L-DOPA in the plasma of a subject over 3 to 7 days, comprising administering, about once every third day to about once weekly, an effective dose of a compound of general formula I as defined above to the subject.

According to another aspect of the invention there is provided a method for decreasing levels of 3-O-methyl-L-DOPA (3-OMD) in the brain of a subject over 3 to 7 days, comprising administering, about once every third day to about once weekly, an effective dose of a compound of general formula I as defined above to the subject.

According to another aspect of the invention there is provided a method for decreasing levels of 3-OMD in the plasma of a subject over 3 to 7 days, comprising administering, about once every third day to about once weekly, an effective dose of a compound of general formula I as defined above to the subject.

According to another aspect of the invention there is provided a method for increasing bioavailability of L-DOPA in the brain of a subject over 3 to 7 days, comprising administering, about once every third day to about once weekly, an effective dose of a compound of general formula I as defined above to the subject.

According to another aspect of the invention there is provided a method for increasing bioavailability of L-DOPA in the plasma of a subject over 3 to 7 days, comprising administering, about once every third day to about once weekly, an effective dose of a compound of general formula I as defined above to the subject.

The subject being treated with the compound of general formula I may also receive therapy with DOPA precursor and/or an aromatic L-amino acid decarboxylase inhibitor. Such therapy with DOPA precursor and/or AADCi may precede, follow or be simultaneous or concomitant with the treatment with the compound of general formula I.

The present invention also relates to a package comprising a pharmaceutical composition of a compound of the general formula I in combination with instructions to administer said formulation with a dosing regimen having a dosing periodicity ranging from about once every third day to about once weekly.

Materials and Methods

In a double-blind, randomised, placebo-controlled study aimed to investigate the tolerability, pharmacokinetics and pharmacodynamics of the compounds of general formula I, groups of eight young healthy males were administered with placebo, 5 mg, 10 mg, 20 mg or 30 mg compound A in the morning under fasting conditions for 8 days. Within each group (5 mg, 10 mg, 20 mg or 30 mg) 2 individuals were randomised to placebo and 6 to treatment.

The soluble catechol-O-methyltransferase (S-COMT) activity was expressed as the amount of metanephrine (in pmol) formed by the action of the S-COMT of washed erythrocytes, on an epinephrine substrate, per milligram of protein in the sample, per hour.

Blood samples were taken at the following times and used for preparing the washed erythrocytes:

Day 1: pre-dose, and 0.25, 0.5, 0.75, 1, 1.5, 2, 3, 4, 6, 8, 10, 12, and 16 h post-dose
Days 2-7: pre-dose only
Day 8: pre-dose, and 0.25, 0.5, 0.75, 1, 1.5, 2, 3, 4, 6, 8, 10, 12, 16, 24, 36, 48, 72, 96, 120 and 144 h post-dose

After centrifugation and removal of the plasma, the supernatant (uppermost erythrocyte layer) was removed and the tubes placed on ice. A volume of cold 0.9% sodium chloride solution equal to double that of the erythrocytes was then added. The erythrocytes were centrifuged and washed using this procedure three times. Centrifugation was undertaken at 4° C. and at approximately 1500 g for 10 minutes. Two accurately 500 μL pipetted aliquots of washed erythrocytes were prepared and each aliquot was stored in a 2-mL tube at −70° C. until required for analysis.

Determination of S-COMT activity was carried out in compliance with Good Laboratory Practices (GLP) by HPLC with electrochemical detection.

### DESCRIPTION OF FIGURES

FIG. 1 shows the mean S-COMT activity [expressed as the % of change of the metanephrine formed in relation to baseline (pre-dose of Day 1)] versus time profile following the first dose (Day 1) and the last dose (Day 8), and at pre-dose of an 8-day period of administration of placebo, 5 mg, 10 mg, 20 mg or 30 mg of Compound A (n=6 for each treatment group and n=8 for each placebo group).

### RESULTS

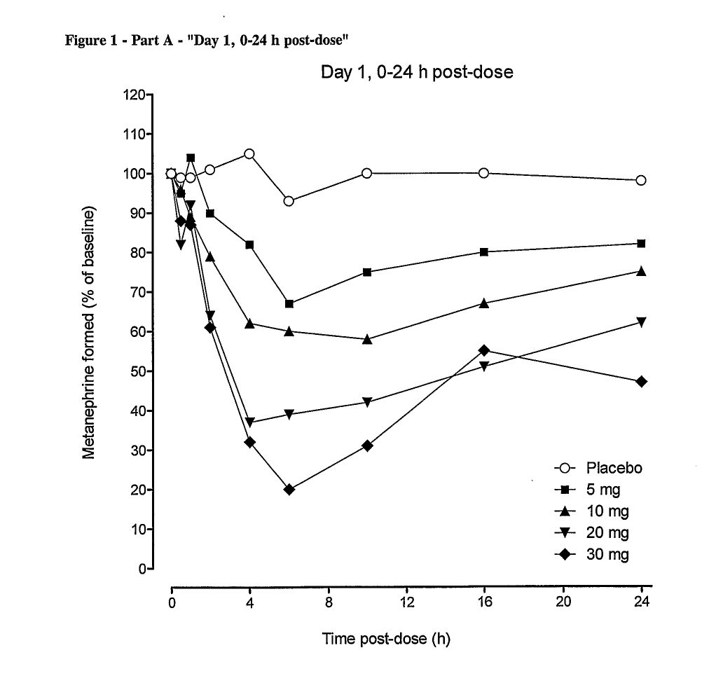

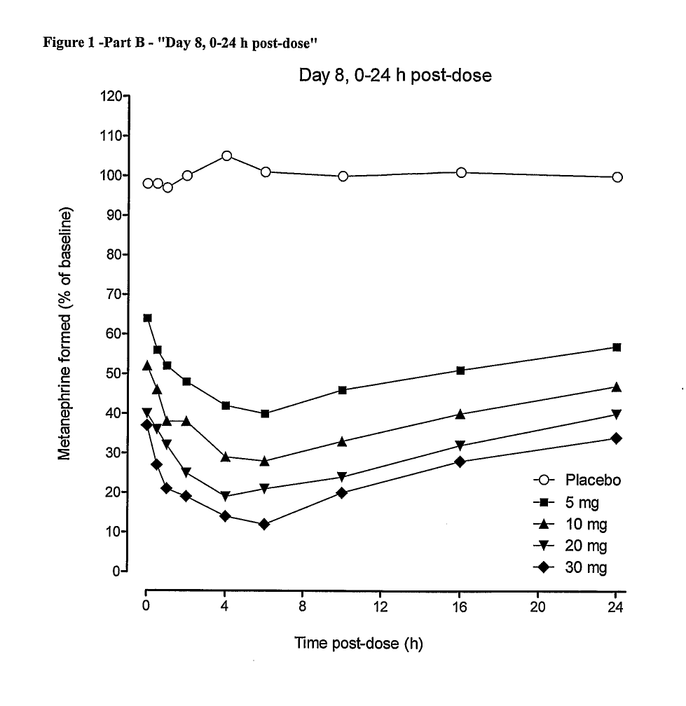

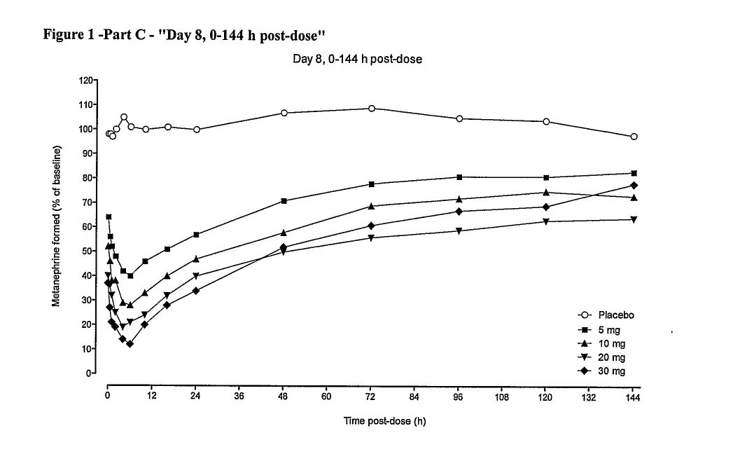

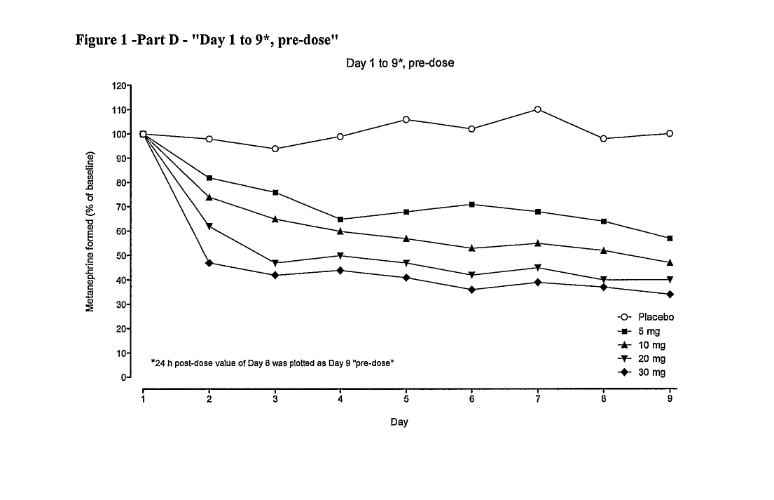

FIG. 1 shows the mean S-COMT activity-time profile following the first dose (FIG. 1A) and the last dose (FIGS. 1B and 1C), and at pre-dose (FIG. 1D) of an 8-day once daily regime with placebo, 5 mg, 10 mg, 20 mg or 30 mg dosage of compound A (n=6 for the treatment group and n=8 for the placebo group).

Marked and sustained S-COMT inhibition was observed from the first administration and 5 days (144 h) after the last dose relevant COMT inhibition was still seen, supporting the possibility of a once weekly administration regime.

The invention will now be described with reference to the following example of preparation, which is not intended to limit the invention in any way.

### Example 1

### Preparation of Compound A

### (5-[3-(2,5-Dichloro-4,6-dimethyl-1-oxy-pyridin-3-yl)-[1,2,4]oxadiazol-5-yl]-3-nitrobenzene-1,2-diol)

a) To a stirred solution of 3,4-dibenzyloxy-5-nitrobenzoic acid (0.50 g, 1.319 mmol) in dimethylformamide (5 mL) at room temperature was added 1,1-carbonyldiimidazole (0.24 g, 1.45 mmol) in one portion. After stirring for ninety minutes, 2,5-dichloro-N′-hydroxy-4,6-dimethylnicotinamide (0.40 g, 1.45 mmol) was added in one portion. The resulting mixture was stirred at 135° C. for five hours and then at room temperature overnight. The reaction mixture was poured onto ice-2 N HCl (100 mL) and the resulting precipitate was filtered off, washed with water and dried in air. Recrystallisation from isopropanol gave a pale yellow solid (0.55 g, 72%).

b) To a stirred solution of the solid obtained above (0.50 g, 0.866 mmol) in dichloromethane (20 mL) was added urea-hydrogen peroxide addition complex (0.41 g, 4.33 mmol) in one portion. The mixture was cooled in an ice-water bath and trifluoroacetic anhydride (0.73 g, 3.46 mmol) was added dropwise. The reaction mixture was allowed to stir at room temperature overnight whereupon insoluble material was filtered off. The filtrate was washed with water and brine, dried over anhydrous magnesium sulphate, filtered and evaporated. The residue was crystallised from isopropanol to give a pale yellow solid (0.35 g, 68%).

c) To a stirred solution of the solid obtained above (0.30 g, 0.5 mmol) in dichloromethane (10 mL) at −78° C. under argon was added boron tribromide (0.38 g, 1.5 mmol) dropwise. The resulting purple suspension was allowed to stir at room temperature for one hour, then cooled again to −78° C. and carefully quenched by the addition of water. After stirring at room temperature for one hour, the precipitate was filtered off, washed with water and dried at 50° C. under vacuum to afford the desired compound as yellow crystals (0.18 g, 87%) of m.p. 237-240° C.

### Example 2

### Pharmaceutical Formulation

Pharmaceutical formulations are prepared as follows:

### Capsule:

| | |
|---|---|
| Compound A | 15.0% |
| Lactose monohydrate | 43.0% |
| Microcrystalline cellulose | 30.0% |
| Povidone | 4.0% |
| Croscarmellose sodium | 5.0% |
| Talc | 2.0% |
| Magnesium stearate | 1.0% |

### Capsule:

| | |
|---|---|
| Compound A | 15.0% |
| Microcrystalline cellulose | 72.5% |
| Ethylcellulose | 5.0% |
| Sodium starch glycolate | 6.0% |
| Colloidal Silicon Dioxide | 0.5% |
| Magnesium stearate | 1.0% |

### Tablet:

| | |
|---|---|
| Compound A | 20.0% |
| Microcrystalline cellulose | 25.0% |
| Calcium Phosphate, dibasic dihydrate | 40.0% |
| Povidone | 6.0% |
| Croscarmellose sodium | 6.0% |
| Talc | 2.0% |
| Magnesium stearate | 1.0% |

### Example 3

### Dosing Regimen

Patients are treated once weekly with tablets containing 100 mg of compound of the general formula I to patients who are suffering from Parkinson's disease and who are on L-DOPA therapy. A long-lasting COMT-inhibition and a significant improvement of the clinical picture is observed.

1-14. (canceled)

15. A pharmaceutical composition comprising a compound of formula I 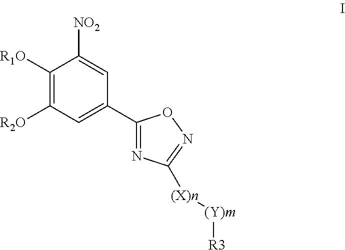
where R₁ and R₂ are the same or different and signify hydrogens, groups hydrolysable under physiological conditions, or optionally substituted alkanoyls or aroyls; X signifies a methylene group; Y represents O, S or NH; n represents 0, 1, 2 or 3; m represents 0 or 1; R₃ signifies a pyridine N-oxide group according to the formula A, B, or C, which is connected as indicated by the unmarked bond: 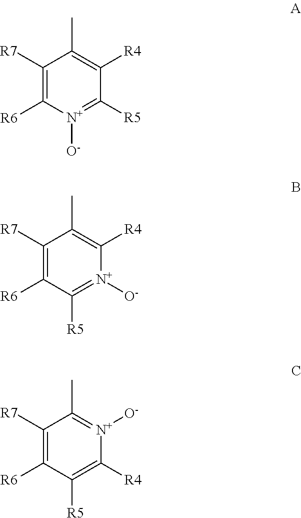
where R₄, R₅, R₆ and R₇ are the same or different, and signify hydrogen, alkyl, thioalkyl, alkoxy, aryloxy, thioaryl, alkanoyl, aroyl, aryl, amino, alkylamino, dialkylamino, cycloalkylamino, heterocycloalkylamino, alkylsulphonyl, arylsulphonyl, halogen, haloalkyl, trifluoromethyl, cyano, nitro or heteroaryl; or two or more of R₄, R₅, R₆ and R₇ taken together signify aliphatic or heteroaliphatic rings or aromatic or heteroaromatic rings; the term ‘alkyl’, including its variant ‘alk-’ in terms such as ‘alkoxy’, ‘alkanoyl’ means carbon residues, straight or branched, containing from one to six carbon atoms; the term ‘aryl’ means a phenyl or naphthyl group; the term ‘heterocycloalkyl’ represents a four to eight-membered cyclic ring optionally incorporating at least one atom of oxygen, sulphur or nitrogen; the term ‘heteroaryl’ represents a five or six-membered ring incorporating at least one atom of sulphur, oxygen or nitrogen; the term ‘halogen’ represents fluorine, chlorine, bromine or iodine; and if R₄, R₅, R₆ and R₇ represent alkyl or aryl they are optionally substituted by one or more hydroxy, alkoxy or halogen groups; or a pharmaceutically acceptable salt or ester thereof, in combination with instructions for the treatment of a central and peripheral nervous system associated disorder to administer said composition according to a dosing regimen having a dosing periodicity ranging from about once every third day to about once every seventh day.

16. The pharmaceutical composition according to claim 15, wherein the dosing regimen is once every third day.

17. The pharmaceutical composition according to claim 15, wherein the dosing regimen is once every fourth day.

18. The pharmaceutical composition according to claim 15, wherein the dosing regimen is once every fifth day.

19. The pharmaceutical composition according to claim 15, wherein the dosing regimen is once every sixth day.

20. The pharmaceutical composition according to claim 15, wherein the dosing regimen is once every seventh day.

21. The pharmaceutical composition according to claim 15, wherein the central and peripheral nervous system associated disorder is treatable with L-DOPA/AADCi therapy.

22. The pharmaceutical composition according to claim 15, wherein the central and peripheral nervous system associated disorder is a movement disorder.

23. The pharmaceutical composition according to claim 22, wherein the movement disorder is Parkinson's Disease.

24. The pharmaceutical composition according to claim 15, wherein the composition further comprises L-DOPA with suitable instructions for its administration.

25. The pharmaceutical composition according to claim 15, wherein the composition further comprises an AADCi with suitable instructions for its administration.

26. The pharmaceutical composition according to claim 25, wherein the AADCi is carbidopa or benserazide.

27. The pharmaceutical composition according to claim 15, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

28. Method of treating at least one pathological state in a patient in need thereof comprising administering to the patient about once every third day to about once every seventh day a pharmacologically effective dose of a compound of formula I 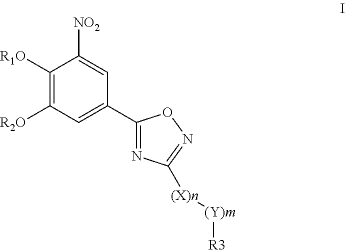
where R₁ and R₂ are the same or different and signify hydrogens, groups hydrolysable under physiological conditions, or optionally substituted alkanoyls or aroyls; X signifies a methylene group; Y represents O, S or NH; n represents 0, 1, 2 or 3; m represents 0 or 1; R₃ signifies a pyridine N-oxide group according to the formula A, B, or C, which is connected as indicated by the unmarked bond: 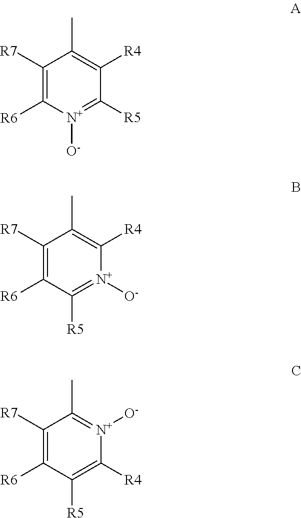
where R₄, R₅, R₆ and R₇ are the same or different, and signify hydrogen, alkyl, thioalkyl, alkoxy, aryloxy, thioaryl, alkanoyl, aroyl, aryl, amino, alkylamino, dialkylamino, cycloalkylamino, heterocycloalkylamino, alkylsulphonyl, arylsulphonyl, halogen, haloalkyl, trifluoromethyl, cyano, nitro or heteroaryl; or two or more of R₄, R₅, R₆ and R₇ taken together signify aliphatic or heteroaliphatic rings or aromatic or heteroaromatic rings; the term ‘alkyl’, including its variant ‘alk-’ in terms such as ‘alkoxy’, ‘alkanoyl’ means carbon residues, straight or branched, containing from one to six carbon atoms; the term ‘aryl’ means a phenyl or naphthyl group; the term ‘heterocycloalkyl’ represents a four to eight-membered cyclic ring optionally incorporating at least one atom of oxygen, sulphur or nitrogen; the term ‘heteroaryl’ represents a five or six-membered ring incorporating at least one atom of sulphur, oxygen or nitrogen; the term ‘halogen’ represents fluorine, chlorine, bromine or iodine; and if R₄, R₅, R₆ and R₇ represent alkyl or aryl they are optionally substituted by one or more hydroxy, alkoxy or halogen groups; or a pharmaceutically acceptable salt or ester thereof.

29. Method as claimed in claim 28 wherein the administration is once every third day.

30. Method as claimed in claim 28 wherein the administration is once every fourth day.

31. Method as claimed in claim 28 wherein the administration is once every fifth day.

32. Method as claimed in claim 28 wherein the administration is once every sixth day.

33. Method as claimed in claim 28 wherein the administration is once every seventh day.

34. Method according to claim 28, wherein the pathological state is a central and peripheral nervous system associated disorder.

35. Method according to claim 28, wherein the condition or disease is treatable with L-DOPA/AADCi therapy

36. Method according to claim 35, wherein the condition or disease is a movement disorder.

37. Method according to claim 36, wherein the movement disorder is Parkinson's Disease.

38. Method according to claim 28, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

39. Method according to claim 28, wherein the method further comprises the sequential or concomitant administration of L-DOPA.

40. Method according to claim 28, wherein the method further comprises the sequential or concomitant administration of an AADCi.

41. Method according to claim 40, wherein the AADCi is carbidopa or benserazide.

42. The pharmaceutical composition according to claim 24 wherein the composition further comprises an AADCi with suitable instructions for administration thereof.

43. The pharmaceutical composition according to claim 24, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

44. The pharmaceutical composition according to claim 25, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

45. The pharmaceutical composition according to claim 42, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

46. The method according to claim 37, wherein the compound of formula I is 5-[3-(2,5-dichloro-4,6-dimethyl-1-oxy-pyridin-3-yl)-[1,2,4]oxadiazol-5-yl]-3-nitrobenzene-1,2-diol.

47. The method according to claim 38, wherein the method further comprises the sequential or concomitant administration of L-DOPA.

48. The method according to claim 46, wherein the method further comprises the sequential or concomitant administration of an AADCi.
